(19) United States
(12) Patent Application Publication
(10) Pub. No.: US 2009/0317328 A1
(43) Pub. Date: Dec. 24, 2009

(54) TRICYCLIC OXAZEPINES AS IN VIVO IMAGING COMPOUNDS

(76) Inventors: Erik Arstad, London (GB); Edward George Robins, London (GB); Yongjun Zhao, London (GB)

Correspondence Address: GE HEALTHCARE, INC., IP DEPARTMENT 101 CARNEGIE CENTER, PRINCETON, NJ, 08540-6231, US

(21) Appl. No.: 12/302,895

(22) PCT Filed: May 31, 2007
(86) PCT No.: PCT/GB2007/002024

§ 371 Date: Aug. 24, 2009

(30) Foreign Application Priority Data
Jun. 2, 2006 (GB) 0610866.6

(51) Int. Cl. A61K 51/04; C07D 267/14; A61P 25/00

(52) U.S. Cl. 424/1.89; 540/550; 424/1.81

(57) ABSTRACT

Novel compounds of formula (I): suitable for use as in vivo imaging agents are provided as well as precursors suitable for the preparation of said compounds. The present invention also provides pharmaceuticals comprising the compounds and kits for the preparation of the pharmaceuticals. Furthermore, use of the compounds for imaging peripheral benzodiazepine receptors in a subject is provided, in particular for imaging pathological conditions in which PBR are upregulated, e.g. Parkinson's disease, multiple sclerosis, Alzheimer's disease and Huntington's disease, neuropathic pain, arthritis, asthma, atherosclerosis and cancer.

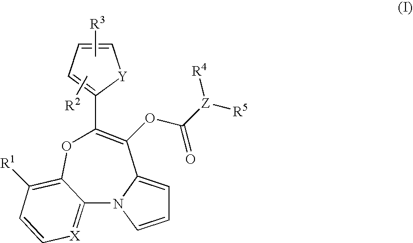

### TECHNICAL FIELD OF THE INVENTION

The present invention relates to the field of medical imaging, and in particular to imaging of disease states associated with the upregulation of peripheral benzodiazepine receptors (PBR). Compounds and methods are provided that are useful for imaging such disease states.

### DESRIPTION OF RELATED ART

Neuroinflammation (NI) incorporates a wide spectrum of complex cellular responses that include activation of microglia and astrocytes and elaboration of cytokines and chemokines, complement proteins, acute phase proteins, oxidative injury, and related molecular processes. These events may have detrimental effects on neuronal function, leading to neuronal injury with, consequently, further glial activation and ultimately neurodegeneration. NI plays an important role in disorders as diverse as Alzheimer's disease, multiple sclerosis (MS), the neurological complications of AIDS, spinal cord injury, some peripheral neuropathies and neurodegenerative disorders, and myositis.

Peripheral benzodiazepine receptors (PBR) are thought to be implicated in NI. PBR are mainly localised in peripheral tissues and glial cells but their physiological function remains to be clearly elucidated. Their presence on the outer membrane of mitochondria indicates a potential role in the modulation of mitochondrial function and in the immune system. It has furthermore been postulated that PBR are involved in cell proliferation, steroidogenesis, calcium flow and cellular respiration. Altered expression of PBR has been observed in a variety of conditions including acute and chronic stress, anxiety, depression, Parkinson's disease, Alzheimer's disease, brain damage, cancer [Gavish et al 1999 Pharm. Rev. 51 p 629], Huntington's disease [Neurosci. Lett. 1998 24(1) pp 53-6], asthma [Gen. Pharmacol. 1997 28(4) pp 495-8], rheumatoid arthritis [Eur. J. Pharmacol. 2002 452(1) pp 111-22], atherosclerosis [J. Nucl. Med. 2004 45 pp 1898-1907] and multiple sclerosis [Banati et al 2000 Brain 123 p 2321]. PBR may also be associated with neuropathic pain, Tsuda et al having observed activated microglia in subjects with neuropathic pain [2005 TINS 28(2) pp 101-7].

Ligands having affinity for PBR are known in the art. A class of indole compounds having affinity for PBR is disclosed in U.S. Pat. No. 6,451,795. The patent states that the compounds are useful for the prevention or treatment of peripheral neuropathies and for the treatment of central neurodegenerative diseases. Okubu et al [Bioorganic & Medicinal Chemistry 2004 12 3569-80] describe the design, synthesis and structure of a group of tetracyclic indole compounds having affinity for PBR, although no particular application of the compounds is discussed. Campiani et al [2002 J. Med. Chem. 45 4276-81] disclose a class of pyrrolobenzoxazepine derivatives that bind to PBR with high affinity, in some cases picomolar affinity. Isoquinoline carboxamide derivatives having affinity for PBR are disclosed in JP 07165721. Radioiodinated and radiobrominated derivatives for in vivo diagnostic applications are also disclosed.

PET imaging using the PBR selective ligand, (R)-[¹¹C]PK11195 provides a generic indicator of central nervous system (CNS) inflammation. Despite the successful use of (R)-[¹¹C]PK11195, it has its limitations. It is known to have high protein binding, and low specific to non-specific binding. The role of its radiolabelled metabolites is not known and quantification of binding requires complex modelling.

An improved imaging agent that specifically targets PBR would be of value for imaging a variety of disease states, as discussed above. A need therefore remains for improved in vivo imaging agents for targeting PBR.

### SUMMARY OF THE INVENTION

The present invention provides novel compounds suitable for use as in vivo imaging agents. Precursors for the preparation of the compounds are also provided, as well as pharmaceuticals comprising the compounds and kits for the preparation of the pharmaceuticals. In a further aspect, the invention provides for use of the compounds for imaging peripheral benzodiazepine receptors in a subject, in particular for imaging conditions in which PBR are thought to be upregulated, e.g. Parkinson's disease, multiple sclerosis, Alzheimer's disease, Huntington's disease, neuropathic pain, arthritis, asthma, atherosclerosis and cancer.

### DETAILED DESCRIPTION OF THE INVENTION

Compounds

In one aspect, the present invention provides a compound of Formula I:

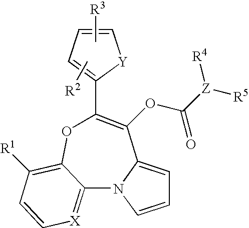

or a salt or solvate thereof, wherein said compound is labelled with an imaging moiety, and wherein:

R¹ is selected from hydrogen, C₁₋₆ alkyl, C₁₋₆ thioalkyl, C₁₋₆ alkoxy, and halogen;

R² and R³ are independently selected from hydrogen, C₁₋₆ alkyl, C¹⁻⁶ thioalkyl, C₁₋₆ alkoxy, and halogen;

R⁴ and R⁵ are independently selected from hydrogen, C₁₋₆ alkyl and C₁₋₆ fluoroalkyl, or together with the group Z to which they are bonded form an optionally-substituted 3-6-membered aliphatic ring optionally containing a heteroatom selected from N, S and O;

X and Z are independently selected from CH and N; and,

Y is selected from O, S, NH, CH═CH, 2-S and N—C₁₋₆ alkyl.

For preferred compounds of Formula I:

R¹ is selected from hydrogen and halogen;

R² and R³ are independently selected from hydrogen, C₁₋₆ alkyl, and halogen;

R⁴ and R⁵ are independently selected from hydrogen and C₁₋₄ alkyl and C₁₋₃ fluoroalkyl, or together with the group Z to which they are bonded form an optionally-substituted 3-6-membered aliphatic ring containing N as a heteroatom;

X is selected from CH or N;

Y is C═C or 2-S, and;

Z is N.

For most preferred compounds of Formula I:

R¹ is hydrogen or Cl;

R² and R³ are independently selected from hydrogen, p-methyl, m-methyl and fluorine;

R⁴ and R⁵ are independently selected from hydrogen, methyl, ethyl and C₁₋₃ fluoroalkyl, or together with the group Z to which they are bonded form cyclopropyl, 4-methyl piperazine or azetidyl,

X is selected from CH or N;

Y is C═C or 2-S, and;

Z is N.

Some especially preferred compounds of the invention are compounds of Formula I wherein:
(i) R¹-R⁴ are hydrogen, R⁵ is ethyl, X is CH, Y is CH═CH and Z is N; or wherein,

(ii) R¹ is chlorine, R²-R⁴ are hydrogen, R⁵ is ethyl, X is CH, Y is CH═CH and Z is N; or wherein,

(iii) R¹-R³ are hydrogen, R⁴ and R⁵ are ethyl, X is N, Y is 2-S and Z is N; or wherein,
(iv) R¹ and R³ are hydrogen, R² is p-methyl, R⁴ and R⁵ are methyl, X is N, Y is CH═CH, and Z is N.

Suitable salts according to the invention, include physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycollic, gluconic, succinic, methanesulphonic, and para-toluenesulphonic acids.

Suitable solvates according to the invention include those formed with ethanol, water, saline, physiological buffer and glycol.

A common starting material to all the compounds of the invention is 5-phenyl-6-oxa-10b-aza-benzo[e]azulen-4-one. The synthesis of this starting material is described by Campiani et al. (J. Med. Chem., 1996, 39, 2672-2680) starting from phenyl-(2-pyrrol-1-yl-phenoxy)-acetic acid, the preparation of which is also described therein.

The term “labelled with an imaging moiety” means either (i) that one of the atoms of the compound of Formula I itself is an imaging moiety, or (ii) that a group comprising an imaging moiety is conjugated to the compound of Formula I.

Imaging Moieties

The “imaging moiety” allows the compound of the invention to be detected using a suitable imaging modality following its administration to a mammalian body in viva. Preferred imaging moieties of the invention are chosen from:
(i) a gamma-emitting radioactive halogen;
(ii) a positron-emitting radioactive non-metal;
(iii) a hyperpolarised NMR-active nucleus;
(iv) a reporter suitable for in vivo optical imaging;
(v) a β-emitter suitable for intravascular detection.

Gamma-Emitting Radioactive Halogens

When the imaging moiety is a gamma-emitting radioactive halogen, the radiohalogen is suitably chosen from ¹²³I, ¹³¹I or ⁷⁷Br. A preferred gamma-emitting radioactive halogen is ¹²³I.

Where the imaging moiety is radioiodine, suitable precursors are those which comprise a derivative which either undergoes electrophilic or nucleophilic iodination or undergoes condensation with a labelled aldehyde or ketone. Examples of the first category are:
(a) organometallic derivatives such as a trialkylstannane (eg. trimethylstannyl or tributylstannyl), or a trialkylsilane (eg. trimethylsilyl) or an organoboron compound (eg. boronate esters or organotrifluoroborates);
(b) a non-radioactive alkyl bromide for halogen exchange or alkyl tosylate, mesylate or triflate for nucleophilic iodination;
(c) aromatic rings activated towards electrophilic iodination (e.g. phenols) and aromatic rings activated towards nucleophilic iodination (e.g. aryl iodonium salt aryl diazonium, aryl trialkylammonium salts or nitroaryl derivatives).

The precursor for radioiodination preferably comprises: a non-radioactive halogen atom such as an aryl iodide or bromide (to permit radioiodine exchange); an activated precursor aryl ring (e.g. a phenol group); an organometallic precursor compound (e.g. trialkyltin, trialkylsilyl or organoboron compound); or an organic precursor such as triazenes or a good leaving group for nucleophilic substitution such as an iodonium salt. Preferably for radioiodination, the precursor comprises an organometallic precursor compound, most preferably trialkyltin.

Precursors and methods of introducing radioiodine into organic molecules are described by Bolton [J. Lab. Comp. Radiopharm., 45, 485-528 (2002)]. Suitable boronate ester organoboron compounds and their preparation are described by Kabalaka et al [Nucl. Med. Biol., 29, 841-843 (2002) and 30, 369-373(2003)]. Suitable organotrifluoroborates and their preparation are described by Kabalaka et al [Nucl. Med. Biol., 31, 935-938 (2004)].

Examples of aryl groups to which radioactive iodine can be attached are given below:

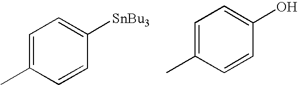

Both contain substituents which permit facile radioiodine substitution onto the aromatic ring. Alternative substituents containing radioactive iodine can be synthesised by direct iodination via radiohalogen exchange, e.g.

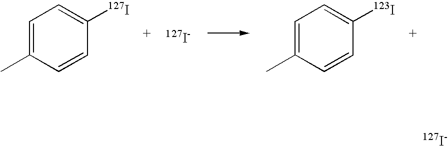

The radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

Positron-Emitting Radioactive Non-Metals

When the imaging moiety is a positron-emitting radioactive non-metal, suitable such positron emitters include: ¹¹C, ¹³N, ¹⁵O, ¹⁷F, ¹⁸F, ⁷⁵Br, ⁷⁶Br or ¹²⁴I. Preferred positron-emitting radioactive non-metals are ¹¹C, ¹³N, ¹⁸F and ¹²⁴I especially ¹¹C and ¹⁸F, most especially ¹⁸F.

When the imaging moiety is a radioactive isotope of fluorine the radiofluorine atom may form part of a fluoroalkyl or fluoroalkoxy group, since alkyl fluorides are resistant to in vivo metabolism. Alternatively, the radiofluorine atom may be attached via a direct covalent bond to an aromatic ring such as a benzene ring. Radiofluorination may be carried out via direct labelling using the reaction of ¹⁸F-fluoride with a suitable chemical group in the precursor having a good leaving group, such as an alkyl bromide, alkyl mesylate or alkyl tosylate. ¹⁸F can also be introduced by alkylation of N-haloacetyl groups with a ¹⁸F(CH₂)₃OH reactant, to give —NH(CO)CH₂O(CH₂)₃¹⁸F derivatives. For aryl systems, ¹⁸F-fluoride nucleophilic displacement from an aryl diazonium salt, aryl nitro compound or an aryl quaternary ammonium salt are suitable routes to aryl-¹⁸F derivatives.

A further approach for radiofluorination as described in WO 03/080544, is to react a precursor compound comprising one of the following substituents:

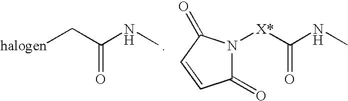

with a compound of Formula V:

¹⁸F—Y*—SH (V)

wherein X* and Y* are each a C₁₋₁₀ hydrocarbyl group optionally including 1-6 heteroatoms;

to give radiofluorinated imaging agents of formula (Va) or (Vb) respectively:

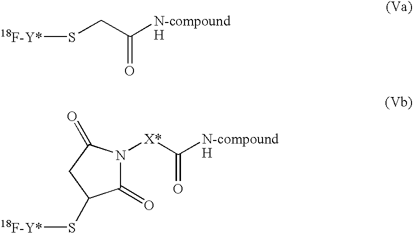

wherein X* and Y* are as defined above, and ‘compound’ is a compound of Formula I, as described above.

A ¹⁸F-labelled compound of the invention may be obtained by formation of ¹⁸F fluorodialkylamines and subsequent amide formation when the ¹⁸F fluorodialkylamine is reacted with a precursor containing, e.g. chlorine, P(O)Ph₃ or an activated ester.

Further details of synthetic routes to ¹⁸F-labelled derivatives are described by Bolton, J. Lab. Comp. Radiopharm., 45, 485-528 (2002).

Where the positron-emitting non-metal is ¹¹C, one approach to labelling with is to react the desmethylated version of a methylated compound precursor with [¹¹C]methyl iodide. It is possible to incorporate ¹¹C by reacting Grignard reagent of the particular hydrocarbon chain of the desired compound with [¹¹C]CO₂. As the half-life of ¹¹C is only 20.4 minutes, it is important that the intermediate ¹¹C moieties have high specific activity and, consequently, are produced using a reaction process which is as rapid as possible.

A thorough review of such ¹¹C-labelling techniques may be found in Antoni et al “Aspects on the Synthesis of ¹¹C-Labelled Compounds” in Handbook of Radiopharmaceuticals, Ed. M. J. Welch and C. S. Redvanly (2003, John Wiley and Sons).

Hyperpolarised NMR-Active Nuclei

When the imaging moiety is a hyperpolarised NMR-active nucleus, such NMR-active nuclei have a non-zero nuclear spin, and include ¹³C, ¹⁵N, ¹⁹F, ²⁹Si and ³¹P. Of these, ¹³C is preferred. By the term “hyperpolarised” is meant enhancement of the degree of polarisation of the NMR-active nucleus over its' equilibrium polarisation. A number of hyperpolarisation methods are known. Certain of these are described by Golman et al [Magn. Reson. Med. 2001, 46, 1-5 and Acad. Radiol. 2002, 9(suppl.2), S507-S510].

The natural abundance of ¹³C (relative to ¹²C) is about 1%. Although it may be possible to carry out hyperpolarisation in a compound containing a natural abundance of the NMR active nuclei, it is preferably enriched with NNR active nuclei before administration. Suitable ¹³C-labelled compounds are suitably enriched to an abundance of at least 5%, preferably at least 50%, most preferably at least 90% before being hyperpolarised. This may include either selective enrichments of one or more sites, or uniform enrichment of all sites. Enrichment can be achieved by chemical synthesis or biological labelling.

Reporter Suitable For In Vivo Optical Imaging

When the imaging moiety is a reporter suitable for in vivo optical imaging, the reporter is any moiety capable of detection either directly or indirectly in an optical imaging procedure. The reporter might be a light scatterer (e.g. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties. Preferred organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Suitable methods for the introduction of a chromophore are detailed in WO 98/048838.

β-Emitter Suitable For Intravascular Detection

When the imaging moiety is a β-emitter suitable for intravascular detection, preferred such β-emitters include the non-metals ³²P, ³³P, ³⁸S, ³⁸Cl, ³⁹Cl ⁸²Br and ⁸³Br.

Preferred Imaging Moieties and Sites For Incorporation

The most preferred imaging moieties of the invention are radioactive, especially gamma-emitting radioactive halogens and positron-emitting radioactive non-metals, particularly those suitable for imaging using SPECT or PET.

The following Formulae Ia-If illustrate preferred sites for the incorporation of an imaging moiety into Formula I, i.e. at any of R¹-R⁵ or at the carbonyl carbon attached to Z. R¹-R⁵ and X, Y and Z are as defined previously for Formula I, and R* either represents an imaging moiety, or a substituent comprising an imaging moiety:

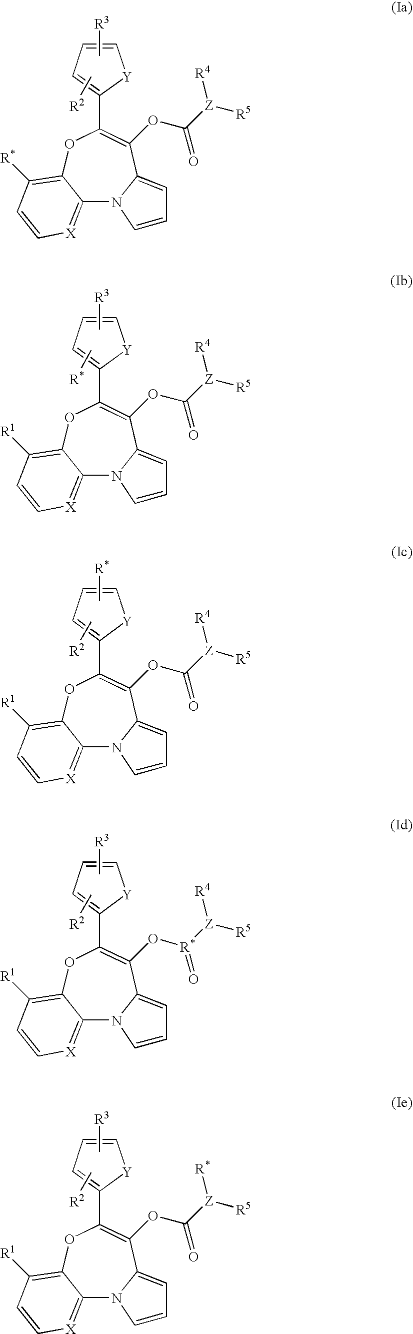

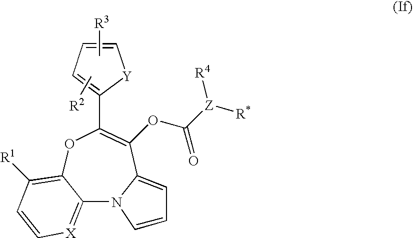

Examples of preferred compounds of Formula I labeled with an imaging moiety are compounds 1 to 6 as follows:

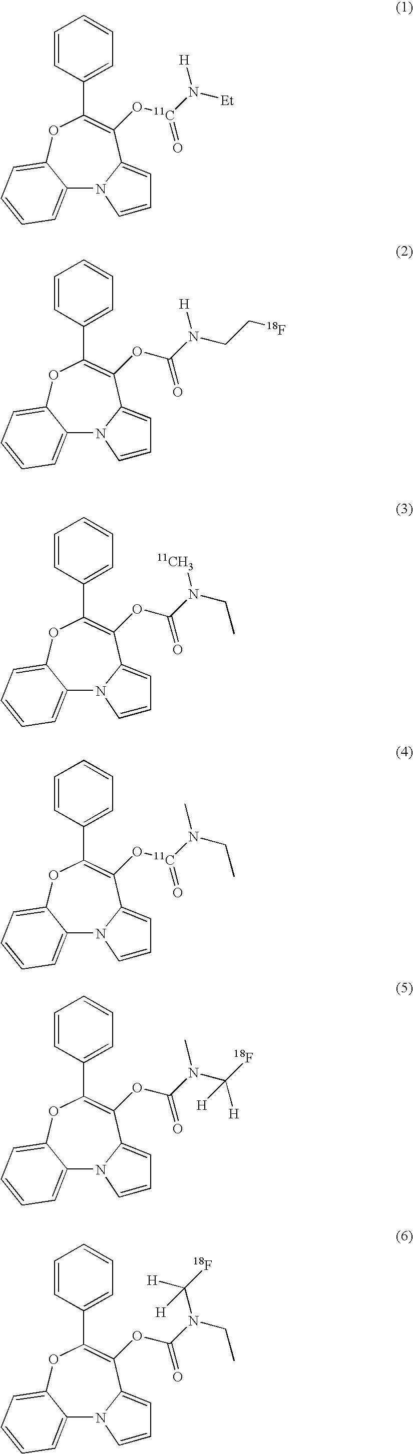

As mentioned above, where the imaging moiety is ¹¹C, a preferred site for incorporation is at the carbonyl group of Formula I (see compounds 1 and 4 above). For example, where R¹-R⁴ are H, R⁵ is as previously defined, X is CH, Y is CH═CH and Z is N, synthesis can start from 5-phenyl-6-oxa-10b-aza-benzo[e]azulen-4-one [Campiani et al. (J. Med. Chem., 1996, 39, 3435)], reaction with a strong base such as an alkali metal hydride (e.g. KH) in an anhydrous solvent (such as tetrahydrofuran) yields a reactive enolate intermediate. Reaction of the enolate with an alkyl-[¹¹C]carbamoyl chloride corresponding to the desired R⁵ group yields the particular compound of the invention.

Another preferred site for incorporation of ¹¹C is as part of a terminal methyl group on R⁴ of Formula I (see compound 3 above). For example, where R¹-R³ are H, R⁴ and R⁵ are as defined previously, X is CH, Y is CH═CH and Z is N, preparation of a desmethyl intermediate, e.g. N-ethyl-carbamic acid 5-phenyl-6-oxa-10b-aza-benzo[e]azulen-4-yl ester, has been described by Campiani et al (J. Med. Chem., 2002, 45, 4276). Reaction with of the particular desmethyl derivative with [¹¹C]methyliodide in the presence of a suitable base such as potassium carbonate in an anhydrous polar solvent (e.g. acetontirile) yields a compound of the invention.

Where the imaging moiety is ¹⁸F, a preferred site of incorporation is at the terminal end of the R⁵ group of Formula I (see compound 2 above). For example, where R¹-R⁴ are H, R⁵ is as defined previously, X is CH, Y is CH═CH and Z is N, synthesis can start from 5-phenyl-6-oxa-10b-aza-benzo[e]azulen-4-one Campiani et al (J. Med. Chem., 2002, 45, 4276). Reaction with a strong base such as an alkali metal hydride (e.g. KH) in an anhydrous solvent (such as tetrahydrofuran) yields the reactive enolate intermediate. Reaction of the enolate with carbamoyl chloride yields the intermediate compound carbamic acid 5-phenyl-6-oxa-10b-aza-benzo[e]azulen-4-yl ester. Reaction in the presence of a suitable base (e.g. potassium carbonate) with an [¹⁸F]fluoroalkyl bromide, corresponding to the desired R⁵ group, in a polar anhydrous solvent (such as acetonitrile) yields the particular compound of the invention. [¹⁸F]Fluoroalkyl bromides may be prepared according to the published procedure of Bauman et al (Tetrahedron Lett., 2003, 44, 9165), or Iwata et al (J. Labelled Compd. Radiopharm., 2003, 46, 555).

Another site for incorporation of ¹⁸F is as part of a [¹⁸F]fluoromethyl group in either R⁴ or R⁵. One route to achieve this where R¹-R⁴ are H, R⁵ is as previously defined, X is CH, Y is CH═CH and Z is N, is to start from 5-phenyl-6-oxa-10b-aza-benzo[e]azulen-4-one. Preparation of the intermediate N-methyl-carbamic acid 5-phenyl-6-oxa-10b-aza-benzo[e]azulen-4-yl ester has been described by Campiani et al (J. Med. Chem., 2002, 45, 4276). Reaction of this intermediate with an [¹⁸F]fluoroalkylbromide, corresponding to the desired R⁵ group, in the presence of a suitable base such as potassium carbonate in an anhydrous polar solvent (e.g. acetontirile) yields the particular compound of the invention. Preparation of [¹⁸F]fluoroalkylbromides may be carried out according to the previously described procedures of Bauman et al or Iwata et al, as mentioned in the previous paragraph. An alternative route to incorporate ¹⁸F is as part of a [¹⁸F]fluoromethyl group in either R⁴ or R⁵ is to react a desmethyl intermediate with [¹⁸F]fluoromethylbromide in the presence of a suitable base such as potassium carbonate in an anhydrous polar solvent (e.g. acetontirile).

Synthetic routes for compounds 1 to 6 are described in more detail in Examples 1 to 6.

Preferably, compounds of the invention do not undergo facile metabolism in vivo, and hence most preferably exhibit a half-life in vivo of 60 to 240 minutes in humans. The compound is preferably excreted via the kidney (i.e. exhibits urinary excretion). The compound preferably exhibits a signal-to-background ratio at diseased foci of at least 1.5, most preferably at least 5, with at least 10 being especially preferred. Where the compound comprises a radioisotope, clearance of one half of the peak level of compound which is either non-specifically bound or free in vivo, preferably occurs over a time period less than or equal to the radioactive decay half-life of the radioisotope of the imaging moiety.

Precursors

In another, the present invention provides a precursor for the preparation of compounds of the invention wherein said precursor is a compound of Formula I derivatised to include a chemical group suitable for labelling with an imaging moiety.

A “precursor” comprises a derivative of the compound of Formula I, designed so that chemical reaction with a convenient chemical form of the imaging moiety occurs site-specifically; can be conducted in the minimum number of steps (ideally a single step); and without the need for significant purification (ideally no further purification), to give the desired imaging agent. Such precursors are synthetic and can conveniently be obtained in good chemical purity. The “precursor” may optionally comprise a protecting group for certain functional groups of the compound of Formula I.

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tetrabutyldimethylsilyl. For thiol groups, suitable protecting groups are: trityl and 4-methoxybenzyl. The use of further protecting groups are described in ‘Protective Groups in Organic Synthesis’, Theorodora W. Greene and Peter G. M. Wuts, (Third Edition, John Wiley & Sons, 1999).

Suitably, the precursor of the invention is derivatised with a chemical group selected from:
(i) an organometallic derivative such as a trialkylstannane or a trialkylsilane;
(ii) a derivative containing an alkyl halide, alkyl tosylate or alkyl mesylate for nucleophilic substitution;
(iii) a derivative containing an aromatic ring activated towards nucleophilic or electrophilic substitution; and
(iv) a derivative which alkylates thiol-containing compounds to give a thioether-containing product.

The following Formulae Ii-Iv illustrate preferred sites for the incorporation of a chemical group into Formula I, wherein R¹-R⁵ and X, Y and Z are as defined previously for Formula I, and CG represents said chemical group:

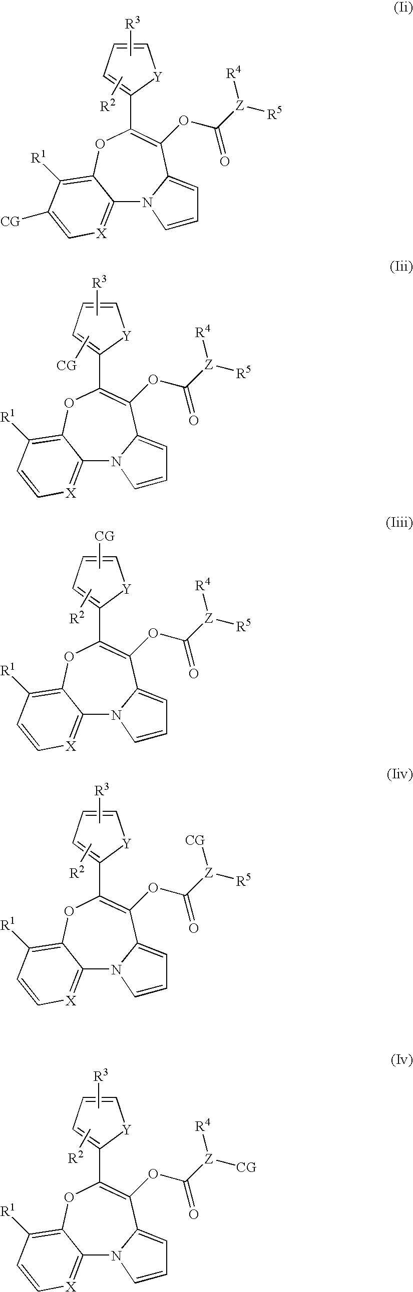

Examples of preferred precursor compounds of the invention are as follows:

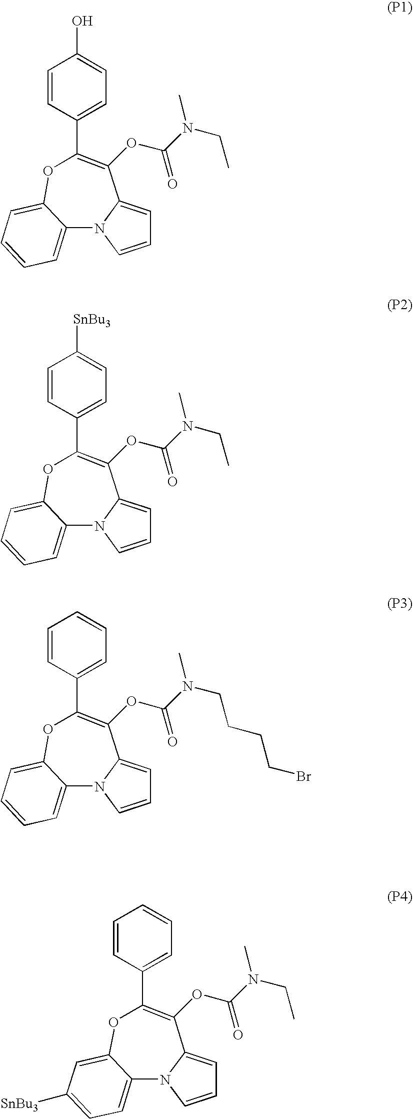

Pharmacueutical Composition

In a further aspect, the present invention provides a pharmaceutical composition which comprises the compound of the invention together with a biocompatible carrier in a form suitable for mammalian administration. Preferably, the pharmaceutical composition is a radiopharmaceutical composition, i.e. the compound of Formula I comprises a radioactive imaging moiety.

The “biocompatible carrier” is a fluid, especially a liquid, in which the compound is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Such pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. For radiopharmaceutical, compositions, the pre-filled syringe may optionally be provided with a syringe shield to protect the operator from radioactive dose. Suitable such radiopharmaceutical syringe shields are known in the art and preferably comprise either lead or tungsten.

The radiopharmaceuticals may be administered to patients for SPECT or PET imaging in amounts sufficient to yield the desired signal, typical radionuclide dosages of 0.01 to 100 mCi, preferably 0.1 to 50 mCi will normally be sufficient per 70 kg bodyweight.

The pharmaceuticals of the present invention may be prepared from kits, as is described below. Alternatively, the pharmaceuticals may be prepared under aseptic manufacture conditions to give the desired sterile product. The pharmaceuticals may also be prepared under non-sterile conditions, followed by terminal sterilisation using e.g. gamma-irradiation, autoclaving, dry heat or chemical treatment (e.g. with ethylene oxide). Preferably, the pharmaceuticals of the present invention are prepared from kits, as described in more detail below.

Kits

A further aspect of the present invention provides kits for the preparation of the pharmaceutical compositions of the third embodiment. Such kits comprise a suitable precursor of the invention, preferably in sterile non-pyrogenic form, so that reaction with a sterile source of an imaging moiety gives the desired pharmaceutical with the minimum number of manipulations. Such considerations are particularly important for radiopharmaceuticals, in particular where the radioisotope has a relatively short half-life, and for ease of handling and hence reduced radiation dose for the radiopharmacist. Hence, the reaction medium for reconstitution of such kits is preferably a “biocompatible carrier” as defined above, and is most preferably aqueous.

Suitable kit containers comprise a sealed container which permits maintenance of sterile integrity and/or radioactive safety, plus optionally an inert headspace gas (e.g. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired e.g. to change the headspace gas or degas solutions.

In the case of precursors bound to a solid phase, the sealed container may be a cartridge provided as part of the kit, which can be plugged into a suitably adapted automated synthesizer. The cartridge may contain, apart from the solid support-bound precursor, a column to remove unwanted reactants, and an appropriate vessel connected so as to allow the reaction mixture to be evaporated and allow the product to be formulated as required. These cartridges are especially useful for the preparation of compounds of the invention labeled with short-lived radioisotopes such as ¹¹C or ¹⁸F.

Preferred aspects of the precursor when employed in the kit are as described above. The precursors for use in the kit may be employed under aseptic manufacture conditions to give the desired sterile, non-pyrogenic material. The precursors may also be employed under non-sterile conditions, followed by terminal sterilisation using e.g. gamma-irradiation, autoclaving, dry heat or chemical treatment (e.g. with ethylene oxide). Preferably, the precursors are employed in sterile, non-pyrogenic form. Most preferably the sterile, non-pyrogenic precursors are employed in the sealed container as described above.

The kits may optionally further comprise additional components such as a radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation. The “biocompatible cation” and preferred embodiments thereof are as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservatives) of the present invention is to inhibit the growth of any such micro-organism in the pharmaceutical composition post-reconstitution, i.e. in the imaging product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservatives) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the precursor is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

Imaging Methods

The compounds of the invention are useful for in vivo imaging. Accordingly, in a further aspect, the present invention provides a compound of the invention for use in an in vivo imaging method, e.g. SPECT or PET. The imaging method may be used to study PBR in healthy subjects, or in subjects known or suspected to have a pathological condition associated with abnormal expression of PBR (a “PBR condition”). Preferably, said method relates to the in vivo imaging of a subject suspected to have a PBR condition, and therefore has utility in the diagnosis of said condition. Examples of such conditions include neuropathologies such as Parkinson's disease, multiple sclerosis, Alzheimer's disease and Huntington's disease where neuroinflammation is present. Other PBR conditions that may be imaged with the compounds of the invention include neuropathic pain, arthritis, asthma, atherosclerosis and cancer. Most preferably, said imaging method relates to the in vivo imaging of a subject suspected to have a PBR condition where neuroinflammation is present.

This aspect of the invention also provides a method for the in vivo diagnosis or imaging in a subject of a PBR condition, comprising administration of a pharmaceutical composition comprising a compound of the invention. Said subject is preferably a mammal and most preferably a human. In an alternative embodiment, this aspect of the invention furthermore provides for the use of the compound of the invention for imaging in vivo in a subject of a PBR condition wherein said subject is previously administered with the pharmaceutical composition of the invention.

By “previously administered” is meant that the step involving the clinician, wherein the imaging agent is given to the patient e.g., intravenous injection, has already been carried out. This aspect of the invention includes the use of the imaging agent of the first embodiment for the manufacture of diagnostic agent for the diagnostic imaging in vivo of a PBR condition.

Furthermore, this aspect of the invention provides for use of the compound of the invention in the manufacture of a pharmaceutical for the in vivo diagnosis or imaging of a PBR condition.

Treatment Monitoring

In a yet further aspect, the invention provides a method of monitoring the effect of treatment of a human or animal body with a drug to combat a PBR condition, said method comprising administering to said body a compound of the invention and detecting the uptake of said compound, said administration and detection optionally but preferably being effected repeatedly, e.g. before, during and after treatment with said drug.

### BRIEF DESCRIPTION OF THE EXAMPLES

Examples 1-6 describe synthesis of compounds 1-6 of the present invention, all of which are PET imaging agents.

### Examples

### Example 1

### Synthesis of Ethyl-[¹¹C]carbamic acid 5-phenyl-6-oxa-10b-aza-benzo[e]azulen-4-yl ester

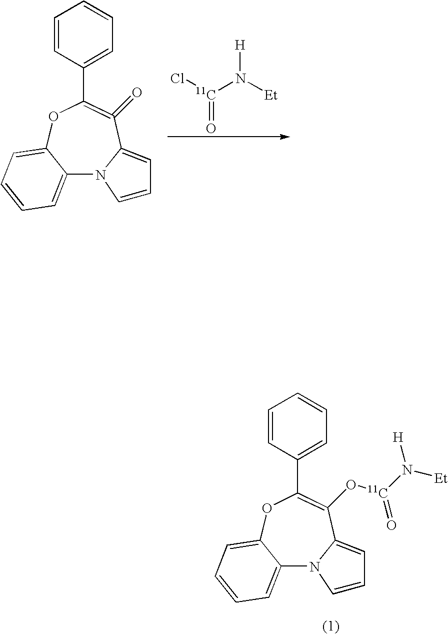

By analogy with the procedure described by Campiani et al (J. Med. Chem., 1996, 39, 3435), starting from 5-phenyl-6-oxa-10b-aza-benzo[e]azulen-4-one, reaction with a strong base such as an alkali metal hydride (e.g. KH) in an anhydrous solvent (such as tetrahydrofuran) yields the reactive enolate intermediate. Reaction of the enolate with ethyl-[¹¹C]carbamoyl chloride yields the desired ethyl-[¹¹C]carbamic acid 5-phenyl-6-oxa-10b-aza-benzo[e]azulen-4-yl ester (1).

Ethyl-[¹¹C]carbamoyl chloride is prepared by a similar route to other reported [¹¹C]carbamoyl chlorides (see for example Lidstroem et al, J. Labelled Compd. Radiopharm., 1997, 40, 788). Reaction of [¹¹C]phosgene with a solution of ethylamine in an anhydrous solvent such as THF yields the desired that Ethyl-[¹¹C]carbamoyl chloride.

### Example 2

### Synthesis of N-[¹⁸F]fluoroethylcarbamic acid 5-phenyl-6-oxa-10b-aza-benzo[e]azulen-4-yl ester

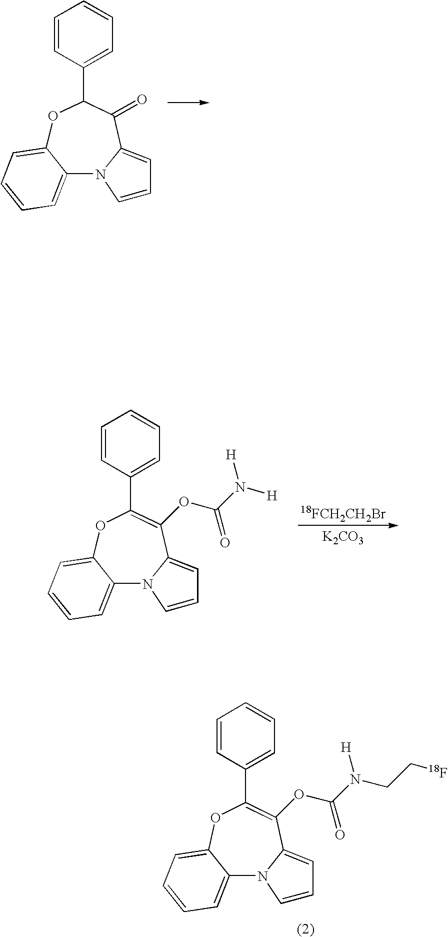

Starting from 5-phenyl-6-oxa-10b-aza-benzo[e]azulen-4-one, reaction with a strong base such as an alkali metal hydride (e.g. KH) in an anhydrous solvent (such as tetrahydrofuran) yields the reactive enolate intermediate. Reaction of the enolate with carbamoyl chloride yields carbomic acid 5-phenyl-6-oxa-10b-aza-benzo[e]azulen-4-yl ester. Reaction of this ester in the presence of potassium carbonate with [¹⁸F]fluoroethyl bromide in acetonitrile yields the desired N-[¹⁸F]fluoroethylcarbamic acid 5-phenyl-6-oxa-10b-aza-benzo[e]azulen-4-yl ester (2).

[¹⁸F]Fluoroethyl bromide may be prepared according to the published procedure of Bauman et al (Tetrahedron Lett., 2003, 44, 9165).

### Example 3

### Synthesis of N-ethyl-N-[¹¹C]methyl-carbamic acid 5-phenyl-6-oxa-10b-aza-benzo[e]azulen-4-yl ester

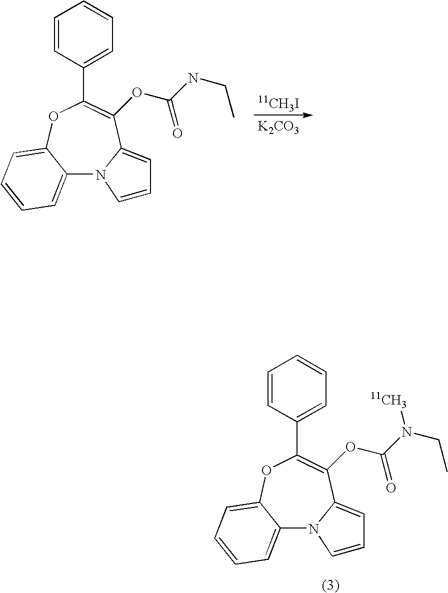

Starting from 5-phenyl-6-oxa-10b-aza-benzo[e]azulen-4-one, the preparation of the desmethyl labeling precursor N-ethyl-carbamic acid 5-phenyl-6-oxa-10b-aza-benzo[e]azulen-4-yl ester has been described by Campiani et al (J. Med. Chem., 2002, 45, 4276). Reaction of this ester with [¹¹C]methyliodide in the presence of potassium carbonate in acetontirile yields the desired N-ethyl-N-[¹¹C]methyl-carbamic acid 5-phenyl-6-oxa-10b-aza-benzo[e]azulen-4-yl ester (3).

### Example 4

### Synthesis of N-ethyl-N-methyl-[¹¹C]carbamic acid 5-phenyl-6-oxa-10b-aza-benzo[e]azulen-4-yl ester

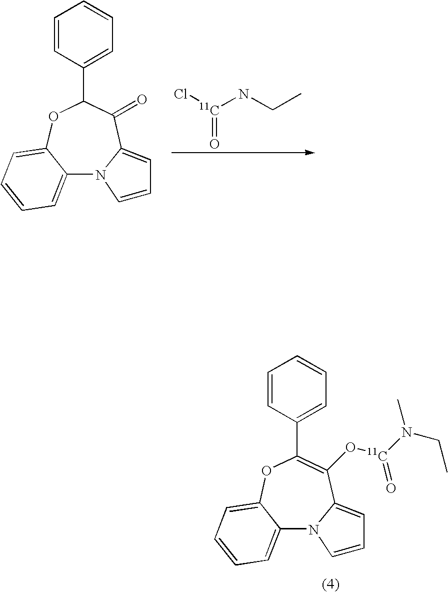

By analogy with Example 1, starting from 5-phenyl-6-oxa-10b-aza-benzo[e]azulen-4-one, reaction with a strong base such as an alkali metal hydride (e.g. KH) in an anhydrous solvent (such as tetrahydrofuran) yields the reactive enolate intermediate. Reaction of the enolate with N-ethyl-N-methyl-[¹¹C]carbamoyl chloride will yield the desired N-ethyl-N-methyl-[¹¹C]carbomic acid 5-phenyl-6-oxa-10b-aza-benzo[e]azulen-4-yl ester (4).

N-Ethyl-N-methyl-[¹¹C]carbamoyl chloride may be prepared by a similar route to other reported [¹¹C]carbamoyl chlorides (see for example Lidstroem et al, J. Labelled Compd. Radiopharm., 1997, 40, 788). Reaction of [¹¹C]phosgene with a solution of ethylmethylamine in an anhydrous solvent such as THF yields the desired that N-Ethyl-N-methyl-[¹¹C]carbamoyl chloride.

### Example 5

### Synthesis of N-[¹⁸F]fluoromethyl-N-methyl-carbamic acid 5-phenyl-6-oxa-10b-aza-benzo[e]azulen-4-yl ester

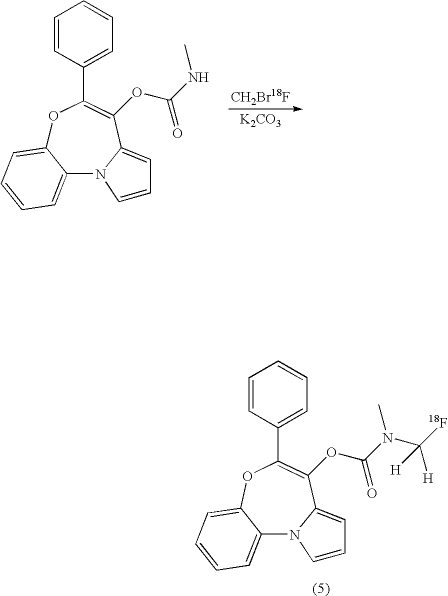

Starting from 5-phenyl-6-oxa-10b-aza-benzo[e]azulen-4-one, the preparation of N-methyl-carbamic acid 5-phenyl-6-oxa-10b-aza-benzo[e]azulen-4-yl ester has been described by Campiani et al (J. Med. Chem., 2002, 45, 4276). Reaction of this ester with [¹⁸F]fluoromethylbromide in the presence of potassium carbonate in acetontirile yields the desired N-[¹⁸F]fluoromethyl-N-methyl-carbamic acid 5-phenyl-6-oxa-10b-aza-benzo[e]azulen-4-yl ester (5).

The preparation of [¹⁸F]fluoromethylbromide is carried out according to the previously described procedure of Iwata et al., (J. Labelled Compd. Radiopharm., 2003, 46, 555).

### Example 6

### Synthesis of N-ethyl-N-[¹⁸F]fluoromethyl-carbamic acid 5-phenyl-6-oxa-10b-aza-benzo[e]azulen-4-yl ester

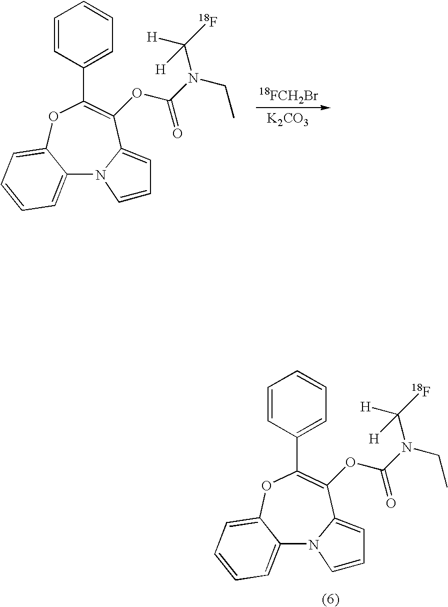

Starting from 5-phenyl-6-oxa-10b-aza-benzo[e]azulen-4-one, the preparation of N-ethyl-carbamic acid 5-phenyl-6-oxa-10b-aza-benzo[e]azulen-4-yl ester has been described by Campiani et al (J. Med. Chem., 2002, 45, 4276). Reaction of this ester with [¹⁸F]fluoromethylbromide in the presence of potassium carbonate in acetontirile will yield the desired N-ethyl-N-[¹⁸F]fluoromethyl-carbamic acid 5-phenyl-6-oxa-10b-aza-benzo[e]azulen-4-yl ester (6).

What is claimed is:

1) A compound of Formula I: 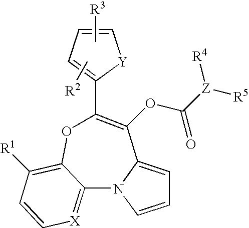
or a salt or solvate thereof, wherein said compound is labelled with an imaging moiety, and wherein:
R¹ is selected from hydrogen, C₁₋₆ alkyl, C₁₋₆ thioalkyl, C₁₋₆ alkoxy, and halogen;
R² and R³ are independently selected from hydrogen, C₁₋₆ alkyl, C₁₋₆ thioalkyl, C₁₋₆ alkoxy, and halogen;
R⁴ and R⁵ are independently selected from hydrogen, C₁₋₆ alkyl and C₁₋₆ fluoroalkyl, or together with the group Z to which they are bonded form an optionally-substituted 3-6-membered aliphatic ring optionally containing a heteroatom selected from N, S and O;
X and Z are independently selected from CH and N; and,
Y is selected from O, S, NH, CH═CH, 2-S, N—C₁₋₆ alkyl.

2) The compound of claim 1 wherein:
R¹ is selected from hydrogen and halogen;
R² and R³ are independently selected from hydrogen, C₁₋₆ alkyl, and halogen;
R⁴ and R⁵ are independently selected from hydrogen, C₁₋₄ alkyl and C₁₋₃ fluoroalkyl, or together with the group Z to which they are bonded form an optionally-substituted 3-6-membered aliphatic ring containing N as a heteroatom;
X is selected from CH or N;
Y is C=C or 2-S, and;
Z is N.

3) The compound of claim 2 wherein:
R¹ is hydrogen or Cl;
R² and R³ are independently selected from hydrogen, p-methyl, m-methyl and fluorine; and,
R⁴ and R⁵ are independently selected from hydrogen, methyl and ethyl and C₁₋₃ fluoroalkyl, or together with the group Z to which they are bonded form cyclopropyl, 4-methyl piperazine or azetidyl.

4) The compound of claim 3 wherein:
(i) R¹-⁴ are hydrogen, R⁵ is ethyl, X is CH, Y is CH═CH and Z is N; or,
(ii) R¹ is chlorine, R²-R⁴ are hydrogen, R⁵ is ethyl, X is CH, Y is CH═CH and Z is N; or,
(iii) R¹-R³ are hydrogen, R⁴ and R⁵ are ethyl, X is N, Y is 2-S and Z is N; or
(iv) R¹ and R³ are hydrogen, R² is p-methyl, R⁴ and R⁵ are methyl, X is N, Y is CH═CH, and Z is N.

5) The compound of claim 1 wherein said imaging moiety is selected from:
(i) a gamma-emitting radioactive halogen;
(ii) a positron-emitting radioactive non-metal;
(iii) a hyperpolarised NMR-active nucleus;
(iv) a reporter suitable for in vivo optical imaging; and, (v) a β-emitter suitable for intravascular detection.

6) The compound of claim 5 wherein said imaging moiety is a gamma-emitting radioactive halogen selected from ¹²³I, ¹³¹I and ⁷⁷Br.

7) The compound of claim 6 wherein said gamma-emitting radioactive halogen is ¹²³I.

8) The compound of claim 5 wherein said imaging moiety is a positron-emitting radioactive non-metal selected from ¹¹C, ¹³N, ¹⁸F and ¹²⁴I.

9) The compound of claim 8 wherein said positron-emitting radioactive non-metal is ¹¹C or ¹⁸F.

10) A precursor for the preparation of the compound of claim 1 wherein said precursor is a compound of Formula I derivatised to include a chemical group suitable for labelling with an imaging moiety, wherein said chemical group comprises:
(i) an organometallic derivative such as a trialkylstannane or a trialkylsilane;
(ii) a derivative containing an alkyl halide, alkyl tosylate or alkyl mesylate for nucleophilic substitution;
(iii) a derivative containing an aromatic ring activated towards nucleophilic or electrophilic substitution;
(iv) a derivative which alkylates thiol-containing compounds to give a thioether-containing product.

11) The precursor of claim 10 wherein said compound of Formula I derivatised to include a chemical group suitable for labeling with an imaging moiety is a compound of any of Formulae Ii-Iv: 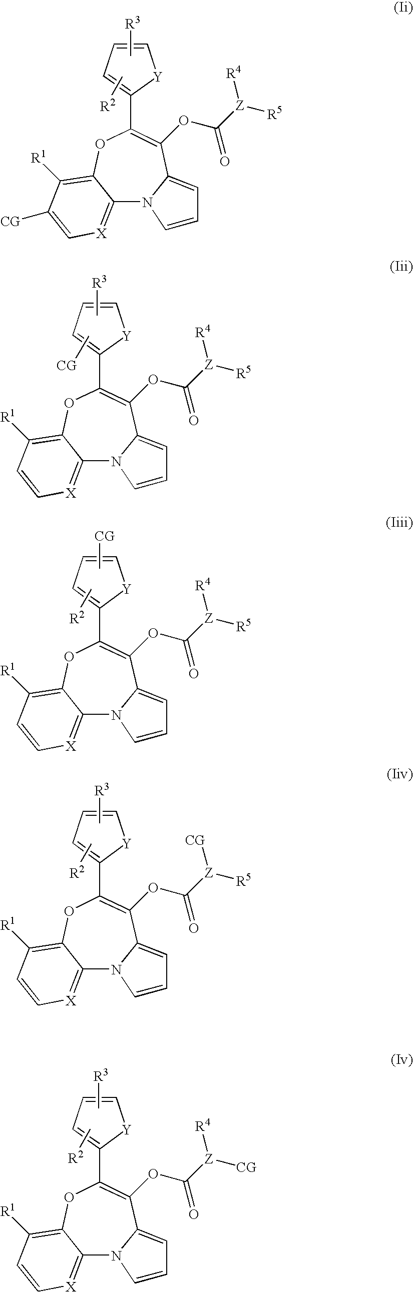
wherein R¹-R⁵ and X, Y and Z are as defined as follows: R¹ is selected from hydrogen, C₁₋₆ alkyl, C₁₋₆ thioalkyl, C₁₋₆ alkoxy, and halogen;
R² and R³ are independently selected from hydrogen, C₁₋₆ alkyl, C₁₋₆ thioalkyl, C₁₋₆ alkoxy, and halogen;
R⁴ and R⁵ are independently selected from hydrogen, C₁₋₆ alkyl and C₁₋₆ fluoroalkyl, or together with the group Z to which they are bonded form an optionally-substituted 3-6-membered aliphatic ring optionally containing a heteroatom selected from N, S and O;
X and Z are independently selected from CH and N; and,
Y is selected from O, S, NH, CH═CH, 2-S, N—C₁₋₆ alkyl, ,and wherein CG represents said chemical group.

12) A pharmaceutical composition which comprises the compound of claim 1 and the salts and solvates thereof together with a biocompatible carrier in a form suitable for mammalian administration.

13) The pharmaceutical composition of claim 12 wherein the compound comprises an imaging moiety which is a radioactive imaging moiety.

14) A kit comprising the precursor of claim 10 wherein said kit is suitable for the preparation of the pharmaceutical composition of a compound that comprises an imaging moiety which is a radioactive imaging moiety of a compound of Formula I: 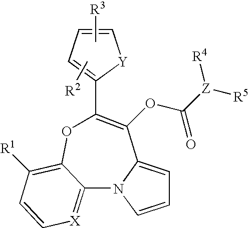
or a salt or solvate thereof, wherein said compound is labelled with an imaging moiety, and wherein:
R¹ is selected from hydrogen, C₁₋₆ alkyl, C₁₋₆ thioalkyl, C₁₋₆ alkoxy, and halogen;
R² and R³ are independently selected from hydrogen, C₁₋₆ alkyl, C₁₋₆ thioalkyl, C₁₋₆ alkoxy, and halogen;
R⁴ and R⁵ are independently selected from hydrogen, C₁₋₆ alkyl and C₁₋₆ fluoroalkyl, or together with the group Z to which they are bonded form an optionally-substituted 3-6-membered aliphatic ring optionally containing a heteroatom selected from N, S and O;
X and Z are independently selected from CH and N; and,
Y is selected from O, S, NH, CH═CH, 2-S, N—C₁₋₆ alkyl.

15) A compound of any of claim 1 for use in an in vivo diagnostic or imaging method.

16) The compound of claim 15 wherein said method is for the in vivo diagnosis or imaging of a PBR condition.

17) A method for the in vivo diagnosis or imaging of a PBR condition in a subject, comprising administration of a pharmaceutical composition comprising a compound of claim 1.

18) Use of the compound of any of claim 1 for imaging in vivo in a subject of a PBR condition wherein said subject is previously administered with the pharmaceutical composition of which comprises the compound of claim 1 and the salts and solvates thereof together with a biocompatible carrier in a form suitable for mammalian administration.

19) Use of the compound of claim 1 in the manufacture of a pharmaceutical for the in vivo diagnosis or imaging of a PBR condition.

20) A method of monitoring the effect of treatment of a human or animal body with a drug to combat a PBR condition, said method comprising administering to said body the pharmaceutical of claim 12, and detecting the uptake of said pharmaceutical.
